(19) United States
(12) Patent Application Publication
(10) Pub. No.: US 2012/0071746 A1
(43) Pub. Date: Mar. 22, 2012

(54) MAGNETIC RESONANCE THERMOMETRY USING PRF SPECTROSCOPY

(76) Inventors: Kobi Vortman, Haifa (IL); David Freundlich, Haifa (IL); Shuki Vitek, Haifa (IL)

(21) Appl. No.: 13/238,743

(22) Filed: Sep. 21, 2011

Related U.S. Application Data
(60) Provisional application No. 61/384,900, filed on Sep. 21, 2010.

(51) Int. Cl. A61B 5/055

(52) U.S. Cl. 600/411; 600/412

(57) ABSTRACT

During the thermal treatment of an anatomical zone of interest, tissue temperature within the zone may be determined with a computational model whose parameters are adjusted using spectroscopy-based temperature measurements at interfaces of fat and non-fat tissues.

### CROSS-REFERENCE TO RELATED APPLICATION

This application claims priority to and the benefit of U.S. Provisional Application No. 61/384,900, filed on Sep. 21, 2010, the entire content of which is hereby incorporated herein by reference.

### FIELD OF THE INVENTION

The present invention relates to magnetic resonance (MR) thermometry, and, in particular, to the use of MR thermometry for monitoring tissue temperature during thermal treatment of internal tissues.

### BACKGROUND OF THE INVENTION

MR imaging of internal body tissues may be used for numerous medical procedures, including diagnosis and surgery. In general terms, MR imaging starts by placing a subject in a relatively uniform, static magnetic field. The static magnetic field causes hydrogen nuclei spins to align with and cause a net magnetization in the general direction of the magnetic field. Radio-frequency (RF) magnetic field pulses are then superimposed on the static magnetic field to flip some of the aligned spins, causing a net magnetization in a plane transverse to the static magnetic field that precesses about the field and thereby induces an RF response signal, called the MR echo or MR response. It is known that different tissues in the subject produce different MR response signals, and this property can be used to create contrast in an MR image. One or more RF receivers detect the duration and strength of the MR response signals, and such data are then processed to generate tomographic or three-dimensional images.

MR imaging can further provide a non-invasive means of quantitatively monitoring in vivo temperatures. This is particularly useful in MR-guided focused ultrasound (MRgFUS) treatment or another MR-guided thermal therapy where the temperature of a treatment area should be continuously monitored in order to assess the progress of treatment and correct for local differences in heat conduction and energy absorption to avoid damage to tissues surrounding the treatment area. The monitoring (e.g., measurement and/or mapping) of temperature with MR imaging is generally referred to as MR thermometry or MR thermal imaging.

Among the various methods available for MR thermometry, the proton resonance frequency (PRF) shift method is often the method of choice due to its linearity with respect to temperature change within non-fatty tissue, its near-independence from the non-fatty-tissue type, and its high spatial and temporal resolution. The PRF shift method is based on the phenomenon that the MR resonant frequency of protons in water molecules changes linearly with temperature. The frequency change is small relative to typical MR center frequencies, only 0.01 ppm/° C. for bulk water and approximately −0.0096 to −0.013 ppm/° C. in non-fatty tissue; however, a frequency shift can also be triggered by magnetic-field instabilities, patient movements, and “susceptibility artifacts” (which occur due to microscopic gradients or variations in magnetic field strength near the interfaces between substances exhibiting different magnetic susceptibilities).

Current approaches to correcting for PRF changes induced by effects other than heating typically exploit the fact that, in many applications, heating triggers an abrupt change in PRF while the other mechanisms mentioned above act more slowly. Thus, short-term changes in temperature can be measured accurately using PRF, but when an absolute temperature measurement is needed over a relatively long period of time (i.e., hours), the effect of slow mechanisms will become pronounced and compromise the accuracy of temperature determinations based on PRF changes.

To determine absolute temperature based on PRF changes over short time periods, a baseline PRF phase image of the region of interest may be acquired at a known temperature prior to heating, and then compared to a second image acquired after the temperature change has occurred. The small observed phase change will be proportional to the change in resonance frequency, and hence to the temperature change, in non-fatty tissue (and will not include a significant contribution from effects unrelated to heating, provided the baseline image is taken shortly before the second image, i.e., immediately prior to treatment). A phase image (or PRF image) may be computed from an MR image, and a temperature-difference map relative to the baseline image may be obtained by (i) determining, on a pixel-by-pixel basis, phase differences between the phase image corresponding to the baseline and the phase image corresponding to a subsequently obtained MR image, and (ii) converting the phase differences into temperature differences based on the PRF temperature dependence while taking into account imaging parameters such as the strength of the static magnetic field and echo time (TE) (e.g., of a gradient-recalled echo). An absolute-temperature map may then be obtained by adding the temperature-differences map to the known temperature distribution prior to treatment (i.e., corresponding to the baseline image), which may, for example, be a uniform temperature of 37° C. throughout the region of interest.

Another class of methods, collectively known as “referenceless thermometry,” is immune to both motion and main-field shifts. Referenceless thermometry does not utilize a separately acquired baseline image, instead deriving a reference phase image from the image portion corresponding to tissue surrounding a heated region by interpolation. While referenceless methods are immune to motion, they are sensitive to rapid anatomical phase variations, which commonly exist at organ edges, since these cannot be accurately expressed as a weighted sum of smooth functions. Further, referenceless methods usually require that the user know the location of the hot spot a priori, so that it can be masked out to avoid bias and temperature underestimation. In addition, in order to determine the absolute temperature in the heated region, the absolute temperature in the area surrounding the heated region needs to be known.

Thus, conventional approaches to PRF thermometry are suitable to map the temperature in an anatomical zone subject to thermal treatment if thermal treatment times are short and the temperature prior to treatment and/or the temperature surrounding a highly localized treated region are known. These conditions break down in many prolonged treatment procedures (e.g., procedures spanning several minutes or hours), for example, when a series of sonications at time intervals that do not suffice for the substantial dissipation of deposited energy results in accumulation of heat in tissue outside the focal zone, or when the interplay between heating of a target region and active cooling of a tissue interface to be protected results in a non-trivial temperature distribution with the zone of interest. Accordingly, there is a need for alternative or supplemental thermometry methods that facilitate mapping absolute temperature in an anatomical region over extended time periods.

### SUMMARY

The present invention overcomes the time constraints inherent in prior PRF techniques by providing systems and methods for monitoring the absolute temperature in an anatomical zone of interest using spectroscopy-based absolute-temperature measurements in certain sub-regions of the zone in conjunction with a computational model that is adjusted based on the measurements. In various embodiments, the invention exploits the fact that PRF, while varying linearly with temperature in non-fatty tissue (which makes PRF-based MR thermometry possible in the first place), is substantially temperature-invariant in fatty tissue. This generally results in two spectral peaks (i.e., resonance peaks at two different frequencies) at locations where fatty and non-fatty tissue are adjacent or mixed. Assuming that the difference in resonant frequencies between fatty and non-fatty tissues at a specific temperature is known, the absolute temperature within a sufficiently small tissue volume containing both fatty and non-fatty tissue can be determined based on the measured difference between the fat and non-fat resonance frequencies. A tissue volume is sufficiently small for this purpose if the temperature is substantially uniform across the volume (e.g., does not vary by a clinically significant amount) and the fatty and non-fatty tissues are close enough so that they are subject to the same magnetic field changes (such that any frequency shift due to magnetic-field rather than temperature changes are subtracted out when the difference between the resonance frequencies is taken).

As used herein, the terms “fat” and “fatty” are meant to characterize tissues, or, more generally, materials, whose PRF response is substantially invariant with temperature, whereas the terms “non-fat” and “non-fatty” are applied to tissues or materials whose PRF response varies substantially linearly with temperature. (By “substantially” is meant within ˜0.01 ppm/° C.). In some embodiments, the conditions for absolute-temperature measurements by means of PRF-spectroscopy are artificially created. For example, if the zone of interest includes the patient's skin, a partially fatty gel pad, i.e., a gel pad that contains a mixture of fatty and non-fatty materials, may be placed in contact with the skin to allow PRF-spectroscopy-based measurements of the temperature in the gel pad and, thus, at the skin (assuming thermal equilibrium between the skin and gel pad). Alternatively, in certain clinical applications, a fat-containing gel pad may be placed adjacent non-fatty tissue (or vice versa) to create a fat/non-fat interface that facilitates determining the absolute temperature at the interface.

In accordance herewith, a computational model (also referred to as a prediction model herein) is used to extend the determination of absolute temperatures into regions where they cannot be measured directly (i.e., tissue regions that include only fatty or only non-fatty tissue). In some embodiments, the computational model includes a differential (or integral) equation that describes the temperature evolution in tissue, taking into account, for example, heat transfer through thermal conduction or blood perfusion, metabolic heat generation, and/or absorption of energy applied to the tissue. The differential equation, supplemented by suitable initial and/or boundary conditions (e.g., a known temperature profile at the beginning of treatment, or a fixed temperature at a boundary of the zone of interest), may be solved numerically (or, in certain cases, analytically) to simulate temperature evolution in the zone of interest, and thereby predict the temperature as a function of time (or at one or more selected discrete points in time). Uncertainties in parameters of the model, such as tissue and blood-flow parameters, can generally result in prediction inaccuracies. In accordance with the present invention, these uncertainties are reduced by adjusting the model parameters based on a comparison of the spectroscopy-based temperature measurements with corresponding temperature predictions for the fat/non-fat interfaces, e.g., using estimation theory or regression to minimize the differences.

The computational model need not necessarily serve to biophysically simulate the temperature evolution in tissue. Rather, in some embodiments, the computational model consists of an analytical temperature profile (e.g., a combination of polynomial or other functions) with adjustable coefficients. In accordance with the present invention, the model coefficients are adjusted to fit the profile to the measurements, i.e., to minimize the error between the measured and predicted temperatures.

Once the computational model has been adjusted based on the temperature measurements in subregions containing (e.g., at interfaces between) fatty and non-fatty tissues or materials, it can be used to compute an absolute-temperature map for the zone of interest. This map may then be used as a temperature baseline in conjunction with conventional PRF-shift thermometry. For example, the temperature change in a focal zone that results from an individual sonication may be determined with traditional reference-based or referenceless thermometry methods, and may be added to a temperature map that reflects the cumulative effect of a series of preceding sonications on the temperature in and surrounding the focal zone, yielding an absolute-temperature map for the entire zone of interest. Supplementing prior PRF techniques with methods according to the present invention can, thus, overcome the time constraints inherent in the prior techniques.

In a first aspect, the present invention provides a method of performing spectroscopy-based magnetic resonance (MR) temperature measurement. In this method, spectroscopy-based temperature measurements are acquired in defined regions along an interface of an anatomic zone of interest. The defined regions may be volumes spanning fatty and non-fatty material, and the spectroscopy-based temperature measurements may include measurements of the proton resonance frequencies in the fatty and non-fatty materials. The volumes may be sufficiently small that a temperature variation through the volumes is not clinically significant. The interface may separate fatty and non-fatty tissues. In some embodiments, the interface includes or consists of a boundary of the anatomic zone of interest, which may separate tissue from a partially fatty gel pad, or non-fatty tissue from a fat-containing gel pad.

The method further includes using a prediction model to computationally predict the temperature in the defined interface regions, adjusting the model based on the temperature measurements, and generating a temperature map of the zone of interest using the adjusted prediction model. Adjusting the model may involve adjusting variable parameters or coefficients of the model, which may include tissue parameters such as, e.g., a perfusion coefficient, a thermal absorption coefficient, or a metabolic heat generation rate. In some embodiments, the prediction model is based on a bioheat transfer equation (e.g., the Pennes equation), which may be numerically solved to generate the temperature map. In other embodiments, the prediction model includes or consists of an analytical temperature profile over the zone of interest, which may be fitted to the temperature measurements. In certain embodiments, the temperature map is used as a baseline for proton-resonance-frequency-shift-based temperature measurements within the zone of interest.

The method may also include subjecting the zone of interest, or a portion thereof, to a temperature-affecting stimulus, such as acoustic energy applied to tissue within the zone of interest (e.g., tissue within the focal zone or the near of far field) or cooling applied to a boundary of the zone of interest.

In another aspect, the invention relates to a system for performing spectroscopy-based magnetic resonance (MR) temperature measurement. The system includes an MRI unit for acquiring spectroscopy-based temperature measurements in defined regions along an interface of an anatomic zone of interest, a storage unit for storing a prediction model and parameters associated therewith, a computer in communication with the MRI unit, and, optionally, a display for displaying the temperature map. The computer is configured to predict the temperature in the defined interface regions using the prediction model, adjust the stored parameters of the prediction model based on the temperature measurements, and generate a temperature map of the zone of interest using the prediction model and the adjusted parameters. The computer may further be configured to cause a thermal-treatment device (such as an ultrasound transducer) to subject at least a portion of the zone of interest to heat, and/or to cause a cooling system to subject at least a portion of a boundary of the zone of interest to cooling. In some embodiments, where the interface comprises a boundary of the zone of interest, the system may further include a fat-containing gel pad for placement against the boundary.

### BRIEF DESCRIPTION OF THE DRAWINGS

The foregoing will be more readily understood from the following detailed description, in particular, when taken in conjunction with the drawings, in which:

FIG. 1 shows an exemplary MRI system in which PRF-thermometry in accordance with the present invention may be implemented;

FIG. 2 schematically illustrates a focused-ultrasound treatment scenario in which fat/non-fat interfaces in the near field facilitate temperature monitoring in accordance with one embodiment;

FIG. 3 illustrates a treatment scenario in which fat-containing gel pads applied to the patient's skin facilitate temperature monitoring in accordance with one embodiment; and

FIG. 4 illustrates a prostate treatment scenario in which fatty tissue at the prostate boundary facilitates temperature monitoring in accordance with one embodiment.

### DETAILED DESCRIPTION

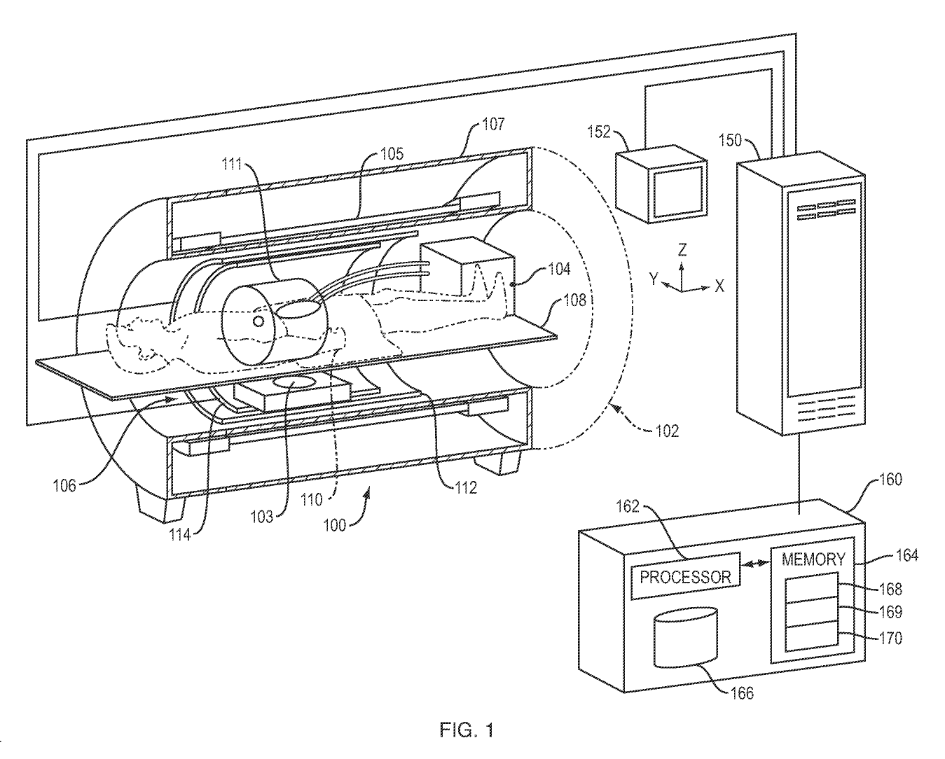

MRI systems in which the techniques described herein may be implemented are well-known in the art; an exemplary system is shown in FIG. 1. The illustrated system 100 comprises an MRI machine 102 and, when an MR-guided thermal procedure is being performed, a thermal therapy device 103 that may be disposed within the bore of the MRI machine 102. The thermal therapy device 103 may be, for example, an ultrasound transducer, an RF or microwave ablation device, a laser, or any other device adapted to heat a target tissue, and may be configured either for placement outside the patient or for insertion into the patient's body. A controller associated with the thermal treatment device 103 may drive the device in accordance with a treatment protocol and/or based MRI data obtained during the treatment procedure. The system 100 may further include an apparatus 104 for actively cooling healthy tissue near the target tissue to avoid damage due to incidental overheating. The cooling apparatus 104 may, for example, include a pump and tubing for circulating a cooling fluid (e.g., water) through a cooling pad in contact with the patient's skin, as well as a controller for adjusting the cooling rate (e.g., based on a sensed or predicted temperature). The controller of the cooling apparatus may be in communication with the controller of the thermal-treatment device such that heating and cooling can be applied in accordance with a desired time sequence.

The MRI machine 102 typically comprises a cylindrical electromagnet 105, which generates a static magnetic field within a bore 106 of the electromagnet 105. The electromagnet 105 may be enclosed in a magnet housing 107. A support table 108, upon which a patient 110 lies during treatment, is disposed within the magnet bore 106. The patient 110 is positioned such that the target tissue, which constitutes the region of interest (ROI), is located within an imaging region 111 in which the static magnetic field is substantially homogeneous. The MRI machine 102 further includes a set of cylindrical magnetic field gradient coils 112, which are typically located within the magnet bore 106, surrounding the patient 110. The gradient coils 112 can generate magnetic field gradients of predetermined magnitudes at predetermined times. Usually, at least three gradient coils 112 that generate magnetic field gradients in three mutually orthogonal directions are provided. Using the field gradients, different spatial locations can be associated with different precession frequencies, thereby giving an MR image its spatial resolution. Further, an RF transmitter coil 114 surrounds the imaging region 111. The RF transmitter coil 114 emits an RF excitation pulse into the imaging region 111, thereby changing the net magnetization of the imaged tissue. The RF transmitter coil 114 may also be used to receive MR response signals emitted from the imaging region 111. Alternatively, the MRI machine 102 may include one or more dedicated RF receiver coils. The MR response signals are amplified, conditioned, digitized into raw data, and converted into arrays of image data using an image-processing system 150, as is known by those of ordinary skill in the art. The image data may then be displayed on a monitor 152, such as a computer CRT, LCD display or other suitable display.

In typical MR imaging procedures, the emission of the RF excitation pulse, the application of the field gradients in various directions, and the acquisition of the RF response signal take place in a predetermined sequence. For example, in some imaging sequences, a linear field gradient parallel to the static magnetic field is applied simultaneously with the excitation pulse to select a slice within the three-dimensional tissue for imaging. Subsequently, time-dependent gradients parallel to the imaging plane may be used to impart a position-dependent phase and frequency on the magnetization vector. Alternatively, an imaging sequence may be designed for a three-dimensional imaging region. Time sequences suitable for PRF thermometry include, for example, gradient-recalled echo (GRE) and spin echo sequences.

In the presence of therapy-induced temperature changes (such as a local temperature increase due to application of focused ultrasound) in non-fatty tissues, variations (such as a “hot spot”) appear in the phase of the image data because the resonance frequency of water protons decreases with increasing temperature. Accordingly, for the purpose of PRF thermometry, the image processing system 150 further includes functionality for extracting phase information from the image data, and computing a map of the temperature-induced relative phase shift based on images acquired before as well as after (or during) heating of the target tissue (i.e., the baseline and treatment images). From the phase-shift map, a map of temperature changes (in units of Δ° C.) may be computed via multiplication with a constant c that is given by:

$$c = \frac{1}{γ α {TEB}_{0} ,}$$

where α is the applicable PRF change coefficient (which is −0.01 ppm/° C. for aqueous tissue), y is the proton gyromagnetic ratio, B₀ is the main magnetic field strength, and TE is the echo time of the GRE or other imaging sequence.

In contrast to aqueous or other non-fatty tissue, the resonance frequency of protons in lipids is largely independent of temperature. At 37° C. (i.e., regular body temperature), the relative difference in resonance frequency between fat and non-fat tissue (i.e., Δf/f, where f is the average of the fat and non-fat resonance frequencies) is about 3.4 ppm. Accordingly, the frequency difference at temperature T can be computed as:

$$Δ f = ( 3.4 ppm + \frac{T - 37 ° C .}{c} ) \cdot f$$

Thus, based on a measured phase (or resonance-frequency) difference between fatty and non-fatty tissues located within the same small volume, the absolute temperature within that volume can be determined.

In various embodiments, the present invention utilizes spectroscopy-based temperature measurements as described above to optimize or improve a computational model for predicting a temperature distribution over the anatomical zone of interest. To implement this functionality, the system 100 typically includes a computer facility 160 including one or more processors 162 in communication with system memory 164 and, optionally, non-volatile data storage 166 (such as a hard drive), which may store the computational model and the values of parameters associated therewith. The computer facility 160 may be, for example, a general-purpose computer programmed with suitable software; but as used herein, the term “computer” refers to any programmable data-processing entity (e.g., a controller, a tablet, a smart phone, dedicated internal circuitry, etc.) capable of performing the computational operations described herein. The software may implement the computational functionality in one or more computationally discrete modules 168, 169, 170. For example, one module may execute instructions to compute a temperature map based on the computational model with a specific set of parameter values; another module may execute instructions to compare measured and computationally predicted temperature values; and a third module may execute instructions to utilize the outputs of the other two modules to estimate optimized model parameters based thereon.

In some embodiments, the Pennes model of heat transfer in perfused tissue, or a modification thereof, is employed. The Pennes model is based on the assumption that the rate of heat transfer between blood and tissue, h_{b}, is proportional to the product of the blood perfusion rate W_{b} (measured in kg/(s m³)) and the difference between the arterial blood temperature Tₐ and the local tissue temperature T(x, y, z): h_{b}=W_{b}C_{b}(Tₐ−T), where C_{b} is the specific heat of blood (measured in J/(K kg)). Adding a heat-transfer contribution due to thermal conduction in the tissue, and taking into account metabolic heat generation at a rate Qₘ (measured in J/(s m³)), the Pennes equation expresses the thermal energy balance for perfused tissue in the following form:

$$ρ C \frac{\partial T}{\partial t} = k ( \frac{{\partial }^{2} T}{\partial {x}^{2}} + \frac{{\partial }^{2} T}{\partial {y}^{2}} + \frac{{\partial }^{2} T}{\partial {z}^{2}} ) + {W}_{b} {C}_{b} ( {T}_{a} - T ) + {Q}_{m} ,$$

where ρ, C, and k are the density, heat capacity, and thermal conductivity (measured in J/(s m K)) of the tissue, respectively. Within a certain type of tissue, the tissue parameters can, for many practical applications, be assumed to be uniform throughout the tissue; however, certain parameters, such as the metabolic heat generation rate, may vary as a function of time. In regions spanning multiple types of tissue, the tissue parameters usually vary also spatially.

To include the influence of external heat sources, such as ultrasound focused into a target region, on the thermal balance, the Pennes equation may be modified by inclusion of an additional term Qₑₓₜ, which is, generally, a function of spatial coordinates and time:

$$ρ C \frac{\partial T}{\partial t} = k ( \frac{{\partial }^{2} T}{\partial {x}^{2}} + \frac{{\partial }^{2} T}{\partial {y}^{2}} + \frac{{\partial }^{2} T}{\partial {z}^{2}} ) + {W}_{b} {C}_{b} ( {T}_{a} - T ) + {Q}_{m} + {Q}_{ext} .$$

In principle, the term Qₑₓₜ may also include the effect of heat sinks (i.e., cooling), as long as the thermal power extracted per unit volume of tissue can be quantified; practically, however, cooling (e.g., applied to the skin) is often more appropriately taken into account via suitable boundary conditions (e.g., a fixed temperature at the skin). Additional modifications to the Pennes equation may be made. For example, for certain applications, metabolic heat generation may be negligible, allowing the equation to be simplified by dropping the term Qₘ.

The (modified) Pennes equation is a partial differential equation of first order in time and second order in space. Solution of the equation, therefore, requires specifying suitable initial and boundary conditions, both of which generally depend on the clinical scenario. For example, for boundaries that coincide with the patient's skin, the temperature at the boundary may be assumed to be equal to the ambient temperature or, if active cooling is applied, the temperature of the cooling fluid. Boundaries inside the body, but sufficiently far away from a thermal-treatment zone, can often be assumed to be at body temperature. Further, the temperature gradient across the respective boundary usually has zero components in directions parallel to the boundary (provided that the temperature does not vary along the boundary); the component perpendicular to the boundary may be determined experimentally or based on further model assumptions. The initial condition typically specifies the temperature distribution at the beginning of the treatment procedure. In the simplest case, the temperature is uniform (e.g., at 37° C.) throughout the region of interest prior to treatment. In other scenarios, e.g., where the region of interest includes the skin (which is at a lower temperature than the rest of the body), the temperature profile may be characterized by a linearly (or non-linearly) decreasing gradient towards the boundary.

In some embodiments, the temperature has reached thermal equilibrium (or near-equilibrium) at the relevant time for which a temperature map is to be computed. For example, during a focused ultrasound procedure, the temperature outside the focal zone tends to stabilize between successive sonications, due to a balance between the deposited heat rate (by focused ultrasound) and heat dissipation (by conduction and perfusion). The temperature in the focal zone may likewise reach equilibrium, or near-equilibrium, during waiting periods in between sonications. For the equilibrium case, the Pennes equation (or its modified version) simplifies to the following steady-state equation (eliminating the need for initial conditions):

$$k ( \frac{{\partial }^{2} T}{\partial {x}^{2}} + \frac{{\partial }^{2} T}{\partial {y}^{2}} + \frac{{\partial }^{2} T}{\partial {z}^{2}} ) + {W}_{b} {C}_{b} ( {T}_{a} - T ) + {Q}_{m} = 0.$$

(The term Qₑₓₜ has been omitted because, in between sonications, the rate of heat deposition by external sources is zero.)

The temperature-dependent or steady-state Pennes equation may generally be solved numerically, using any of a variety of methods known to persons of skill in the art, including, e.g., finite-difference and finite-element methods. In some embodiments, the equation and boundary conditions may be simple enough to allow for a closed analytical solution (i.e., an analytical expression for the temperature distribution that does not involve any approximations (other than those already contained in the model)). Either way, by solving the equation, a temperature map is computed for a given point in time.

The Pennes model is but one bioheat transfer equation. Alternative models include, for example, the continuum model of Chen and Holmes (“Microvascular contributions in tissue heat transfer.” Ann. N.Y. Acad. Sci., vol. 335, pp. 137-150 (1980)), the model of Weinbaum et al. (“Theory and experiment for the effect of vascular microstructure on surface tissue heat-transfer. Part 1. Anatomical foundation and model conceptualization. Part 2. Model formulation and solution.” ASME J. Biomech. Eng., vol. 106, pp. 321-340 (1984)) that takes the existence of large and small blood vessel into account, and the model proposed by Khaled and Vafai (“The role of porous media in modeling flow and heat transfer in biological tissues.” Int. J. Heat Mass Transf., vol. 46, pp. 4989-5003 (2003)), which looks at the tissue as porous medium. These models are described in detail in the scientific literature. The methods described herein may, in general, be applied to any of these models, and a person of skill in the art will be able to select a suitable model for a particular application without undue experimentation.

While heat-transfer models as described above can be used to create a temperature map for the zone of interest, inaccuracies will occur, not only due, potentially, to simplistic model assumptions, but also due to uncertainties in various model parameters, such as, e.g., the quantities k, W_{b}, C_{b}, and Qₘ in the Pennes equation. These parameters may, however, be tuned based on a comparison of predicted with measured temperature values taken at discrete locations along a fat/non-fat interface (to minimize the error). Procedurally, a series of volumes spanning the interface between fatty and non-fatty tissues are defined, and these volumes are scanned for PRF signals. The resonance-frequency difference between the fat and non-fat portion of each defined volume is extracted from the detected spectral signal. Because the PRF signal in the fatty tissue is temperature-invariant and the volume is small relative to the spatial temperature variation, the extracted frequency difference reliably indicates the absolute temperature of the volume. This temperature can be compared with the predicted temperatures, and the model parameters adjusted to reduce or eliminate the error (i.e., so that the predicted temperatures match, as closely as possible, the observed temperatures). (Techniques for estimating model parameters based on empirical values for certain quantities predicted by the model (i.e., in the instant case, the experimentally determined temperatures) are well-known to persons of skill in the art, and can be readily applied to the models and measurement data described herein.)

Applying this technique across a series of volumes provides an accurate map of the temperature along the fat/non-fat tissue boundary, facilitating more widespread (and therefore valid) calibration of the prediction model by varying its parameters. The prediction model, in turn, may be used to provide a reference (baseline) map of temperatures for PRF thermometry. Thus, conventional PRF thermometry may be used to measure temperature changes due to treatment events that occur on short time scales, and the model may serve to monitor the baseline temperature over longer time periods spanning a complex treatment procedure. In some embodiments, the computationally predicted temperature map (particularly as improved by experimental feedback) itself provides sufficient information, and need not be used as a baseline for subsequent PRF-shift imaging of the region. For example, when focused ultrasound is used to ablate or otherwise destroy cancerous tissue, the temperature in the focal zone may not need to be monitored, as long as a temperature above an efficacy threshold can be assumed. However, to avoid damage to surrounding healthy tissue, it may be necessary to track the absolute temperature of that tissue. Based on knowledge of the temperature in the focal zone, the temperature in the healthy tissue can be computed, exploiting the fact the temperature outside the focal zone varies slowly over time as well as space.

The above-described method for adjusting model parameters based on discrete temperature measurements is not limited to biophysical prediction model, but applies, generally, to any computational model suitable for computing a temperature map for the anatomical zone of interest and adjustable via variable model parameters. In some embodiments, the computational model utilizes or consists of an analytical expression for the temperature profile of the zone. The profile may, for example, be constructed from polynomial, exponential, or other sets of functions by linear or non-linear superposition with adjustable coefficients. The coefficients allow fitting the profile to the experimental data (i.e., the measured temperatures at the fat/non-fat interfaces, temperatures at the skin or other tissue boundaries, etc.).

A particular problem to which the invention is suited involves the accumulation of heat in the near and far fields of an energy beam. Specifically, during focused ultrasound treatment, many sonications are applied that are non-overlapping at the target zone, but overlap significantly in the near field and the far field; that is, while the focus of each sonication is spatially distinct, the converging (near-field) and diverging (far-field) beams of the various sonications overlap substantially, causing a slow but non-negligible temperature buildup (e.g., a rise of a couple of degrees that may persist for hours). The problem may be mitigated by an enforced “cooling time” between sonications, but this lengthens the duration of treatment. By establishing the actual absolute temperature in the near and far fields, cooling times can be minimized in order to shorten overall treatment time and improve safety.

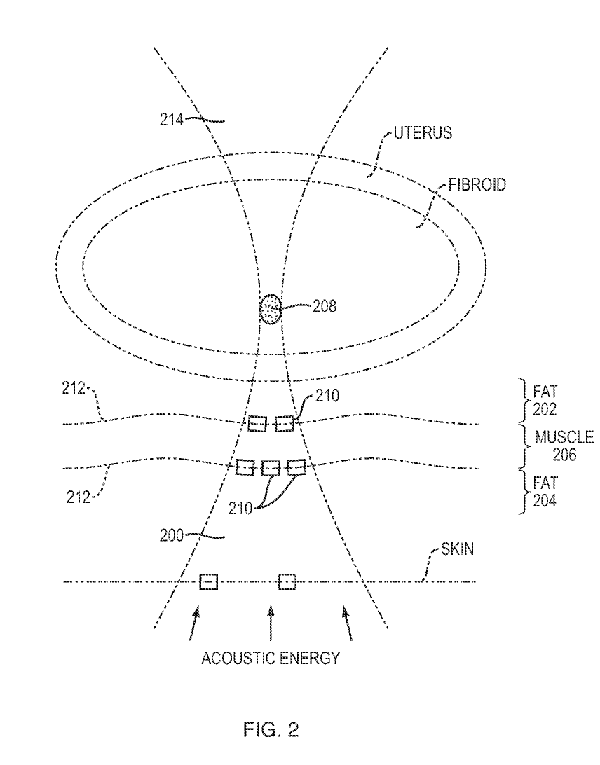

In many anatomic treatment environments, the near-field and/or far-field regions contain fatty areas. For example, as illustrated in FIG. 2, in uterine fibroid treatment, the near field 200 consists of two fat layers 202, 204, one on each side of the peritoneal muscle 206. The temperature evolution outside the focal zone 208 is relatively gradual spatially as well as temporally, and can be modeled and simulated using, for example, the Pennes equation. However, as noted above, the accuracy of the prediction is limited since actual tissue parameters (in particular, the absorption and perfusion coefficients) may be patient-specific and area-specific. To address this problem, small volumes 210 enclosing the interfaces 212 of fat and muscle layers, and extending into each type of tissue, are defined. (These regions are shown in the near field, but the invention is equally applicable to similar boundaries in the far field 214. Moreover, although the volumes 210 are shown as rectangles, they can have any cross-section so long as they span the two adjacent tissue types and encompass enough tissue to permit analysis.) Periodically (e.g., between sonications), a PRF image within the defined volumes 210 is obtained. This may be accomplished either by magnetically exciting the tissue within the defined volumes 210, or by scanning a slice of the entire anatomy that includes the defined volumes 210. Because of the differential frequency response of fat and non-fat tissue, the scan over each volume 210 will exhibit two resonance peaks. Accordingly, the PRF values may be averaged over each volume 210 (i.e., the voxels defining the volumes) for ease of processing, so long as these peaks are not suppressed.

Because the temperature evolves slowly (both temporally and spatially), the selected volumes can be relatively large, especially laterally (e.g., 10×5×10 mm, where the shortest dimension lies along the maximum field gradient), but not so large that there is a clinically significant (e.g., 2° C.) temperature variation over the volume. For the same reason, the scan time can be relatively long (e.g., 30 sec). The larger the volume and the longer the time, the higher the signal-to-noise ratio (“SNR”) will be. The difference between the two peak frequencies in the scan (for fat and non-fat) is obtained for each volume. Due to the temperature invariance of the PRF signal in fatty tissue and the absence of clinically significant temperature variation over the volume (or during the time it takes to perform the MR scan), the difference between the frequency peaks reliably indicates the absolute temperature of the volume. A high SNR translates into sharper peaks and, therefore, more accurate temperature determinations. Accordingly, the system can be designed to permit trade-off between volume size and scanning time, on one hand, and the accuracy of the temperature measurements, on the other hand. The maximum tolerable error range is determined by the application, but usually depends on what is considered clinically significant; in clinical use, errors of ±2° C. are usually acceptable.

The discrete volume temperature measurements are used to adjust the parameters of the temperature-prediction model by comparing the measurements to the predictions, the prediction model being based on the heat resulting from absorbed acoustic energy and possibly active cooling (as reflected in, for example, the parameter Qₑₓₜ) and the dissipation of heat through the tissue (as reflected in, for example, the Pennes parameters k and W_{b}). The model parameters are adjusted so as to minimize the differences between the model prediction and the actual measurement. This may be accomplished, for example, using conventional error-minimization techniques (e.g., iterative linear regression).

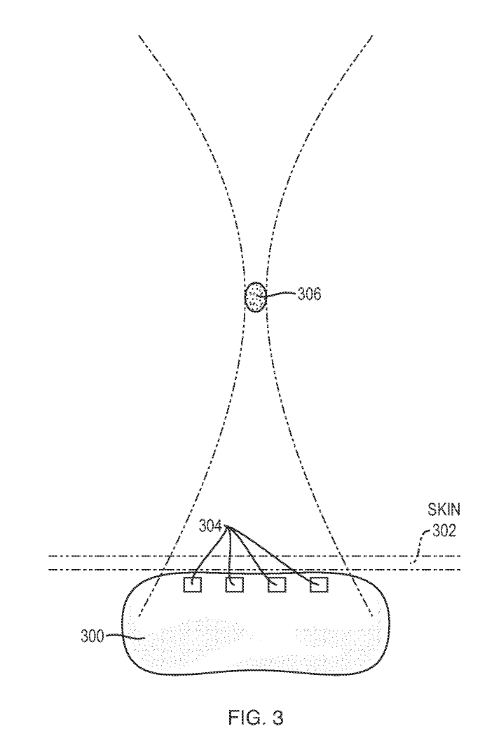

The present invention does not necessarily rely on anatomic structure to provide an interface between fatty and non-fatty tissues, but may utilize artificial mixed fat/non-fat volumes, which may, for example, be employed for temperature measurements taken at the skin. As shown in FIG. 3, a typical focused-ultrasound treatment system utilizes a patient interface that includes a gel pad 300 in contact with the patient's skin 302. For spectral measurements, the gel pad is replaced with a partially fatty gel pad (filled with, e.g., a gelatin suspension containing 10% castor oil or other lipid) that maintains appropriate acoustic properties (e.g., substantial transparency), but facilitates PRF-spectroscopy-based temperature measurement in volumes 304 neighboring the skin 302. These volumes 304 need not include the wall of the gel pad, but should be sufficiently close thereto (e.g., within 1 to 2 mm) to avoid a clinically significant (e.g., >2° C.) temperature difference between the defined volume and the boundary of the gel pad.

In operation, the temperatures of the volumes 304 defined in the gel pad 300 are obtained by MR spectroscopy. The presence of both water and a fatty substance results in two spectral peaks indicative of the absolute temperature, as discussed above. Because these temperatures are identical to those of the underlying skin, they can be used to calibrate the prediction model based on predicted skin temperatures. A temperature map of the region between the ultrasound focus 306 and the skin 302 is then generated using the prediction model. Besides being independent of internal anatomic structure, this approach has also the advantage that the spectroscopic properties of the pad 300—i.e., the relationship between PRF and temperature—can be accurately established in vitro.

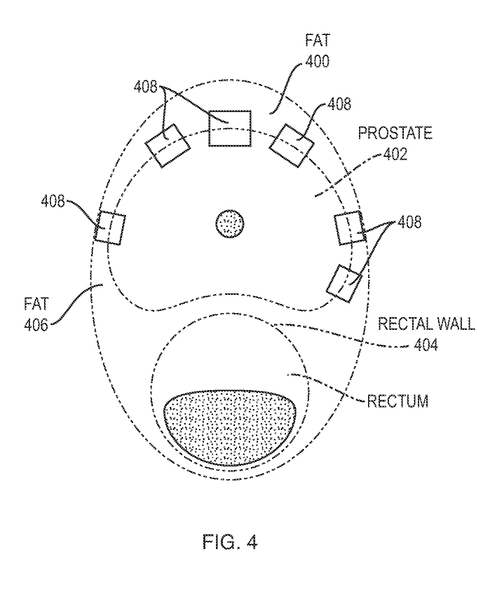

The invention is particularly well suited to monitoring temperature in inhomogeneously cooled treatment domains. Prostate treatment represents an extreme example. In treating the prostate, the rectum may be cooled down (e.g., to 15° C.) while the opposite (anterior) side of the prostate is exposed to body temperature (by heat conduction as well as perfusion). As a result, the prostate tissue stabilizes at an uneven temperature that varies with location. Accordingly, even before acoustic energy is applied, the baseline steady-state temperature profile has significant inhomogeneities, i.e., gradients from the rectal wall to the anterior part of the prostate. FIG. 4 shows an exemplary anatomy of the prostate region. A fatty layer 400 is mostly seen adjacent to the prostate gland 402 at the anterior end (opposite the rectal wall 404). A thinner layer 406 is sometimes seen at the sides of the gland 402. The volumes 408 chosen for spectral temperature measurements are indicated by the rectangular frames in FIG. 4. The interface with the thick fat layer at the far end is expected to provide higher measurement quality, since larger fat volumes correspond to greater PRF temperature invariance and, as a result, a higher SNR. The quality of a measurement in terms of the SNR quality of the peaks on which it is based can be taken into account when tuning the prediction model. In particular, a quality-based weighting can be assigned to temperature measurements so that higher-quality measurements are emphasized as the prediction model is adjusted. That is, as model parameters are tuned based on differences between predicted and measured temperatures, the differences arising from higher-quality measurements are weighted more heavily in the adjustment.

Spectral thermometry measurements are obtained for the defined volumes, mostly at the anterior side of the prostate 402. The boundary temperature at the rectal side can be determined through direct measurement of the temperature of the cooled water. A temperature simulation is then run based on, for example, the Pennes equation operating on the known rectal-side temperature profile to predict temperatures in the defined volumes. As described above, the discrete spectroscopic measurements are compared to what the simulation predicts at the same locations (and with the same spatial averaging over the defined volumes). The model parameters are adjusted so as to minimize the differences. This optimized simulation is now used to create a baseline temperature map over the entire region of interest.

While the foregoing description includes many details and specificities, it is to be understood that these have been included for purposes of explanation only, and are not to be interpreted as limitations of the present invention. It will be apparent to those skilled in the art that other modifications to the embodiments described above can be made without departing from the spirit and scope of the invention. Accordingly, such modifications are considered within the scope of the invention as intended to be encompassed by the following claims and their legal equivalents.

What is claimed is:

1. A method of performing spectroscopy-based magnetic resonance (MR) temperature measurement, the method comprising the steps of:
acquiring spectroscopy-based temperature measurements in defined regions along an interface of an anatomic zone of interest;
using a prediction model, computationally predicting the temperature in the defined interface regions;
adjusting the prediction model based on the temperature measurements; and
computationally generating a temperature map of the zone of interest using the adjusted prediction model.

2. The method of claim 1 wherein the interface separates fatty and non-fatty tissue.

3. The method of claim 1 wherein the interface comprises a boundary of the anatomic zone of interest.

4. The method of claim 3 wherein the boundary separates non-fatty tissue from a fat-containing gel pad.

5. The method of claim 3 wherein the boundary separates tissue from a partially fatty gel pad.

6. The method of claim 1 wherein adjusting the prediction model comprises adjusting variable parameters of the prediction model.

7. The method of claim 6 wherein the variable parameters comprise tissue parameters.

8. The method of claim 6 wherein the tissue parameters comprise at least one of a perfusion coefficient, a thermal absorption coefficient, or a metabolic heat generation rate.

9. The method of claim 1 wherein the prediction model is based on a bioheat transfer equation.

10. The method of claim 9 wherein the bioheat transfer equation is the Pennes equation.

11. The method of claim 9 wherein generating the temperature map comprises numerically solving the bioheat transfer equation.

12. The method of claim 1 wherein the prediction model comprises an analytical temperature profile over the zone of interest.

13. The method of claim 12 wherein adjusting the prediction model comprises fitting the analytical temperature profile to the temperature measurements.

14. The method of claim 1 further comprising subjecting at least a portion of the zone of interest to a temperature-affecting stimulus.

15. The method of claim 14 wherein the stimulus comprises acoustic energy applied to tissue within the zone of interest.

16. The method of claim 15 wherein the tissue includes tissue within at least one of a focal zone, a near field, or a far field.

17. The method of claim 14 wherein the stimulus comprises cooling applied to a boundary of the zone of interest.

18. The method of claim 1 wherein the defined regions are volumes spanning fatty and non-fatty material.

19. The method of claim 18 wherein the volumes are sufficiently small that a temperature variation through the volumes is not clinically significant.

20. The method of claim 18 wherein the spectroscopy-based temperature measurements comprise measurements of the proton resonance frequencies in the fatty and non-fatty materials.

21. The method of claim 1 further comprising using the temperature map as a baseline for proton-resonance-frequency-shift-based temperature measurements within the zone of interest.

22. A system for performing spectroscopy-based magnetic resonance (MR) temperature measurement, the system comprising:
(a) an MRI unit for acquiring spectroscopy-based temperature measurements in defined regions along an interface of an anatomic zone of interest;
(b) a storage unit for storing a prediction model and parameters associated therewith;
(c) a computer, in communication with the MRI unit, configured to:
predict, using the prediction model, the temperature in the defined interface regions;
adjust the stored parameters of the prediction model based on the temperature measurements; and
generate, using the prediction model and the adjusted parameters, a temperature map of the zone of interest.

23. The system of claim 22 further comprising a display for displaying the temperature map.

24. The system of claim 22 wherein the computer is further configured to cause a thermal-treatment device to subject at least a portion of the zone of interest to heat.

25. The system of claim 24 wherein the thermal-treatment device comprises an ultrasound transducer.

26. The system of claim 22 wherein the computer is further configured to cause a cooling system to subject at least a portion of a boundary of the zone of interest to cooling.

27. The system of claim 22 wherein the interface comprises a boundary of the zone of interest, the system further comprising a fat-containing gel pad for placement against the boundary.
